(19) United States
(12) Patent Application Publication
(10) Pub. No.: US 2013/0084614 A1
(43) Pub. Date: Apr. 4, 2013

(54) METHODS OF REMOVING INHIBITORS FROM CELLULOSIC BIOMASS AND PRODUCING ALCOHOLS

(71) Applicants: Dan L. Fanselow, White Bear Lake, MN (US); Masayuki Nakamura, Woodbury, MN (US); Richard B. Ross, Cottage Grove, MN (US); Brady P. Haislet, Maple Plain, MN (US); Alicia A. Petryk, Enfield, NH (US)

(72) Inventors: Dan L. Fanselow, White Bear Lake, MN (US); Masayuki Nakamura, Woodbury, MN (US); Richard B. Ross, Cottage Grove, MN (US); Brady P. Haislet, Maple Plain, MN (US); Alicia A. Petryk, Enfield, NH (US)

(73) Assignee: 3M INNOVATIVE PROPERTIES COMPANY, ST. PAUL, MN (US)

(21) Appl. No.: 13/704,327

(22) PCT Filed: Jun. 14, 2011
(86) PCT No.: PCT/US11/40259

§ 371 Date: Dec. 14, 2012

Related U.S. Application Data
(60) Provisional application No. 61/354,506, filed on Jun. 14, 2010.

(51) Int. Cl. C12P 7/10

(52) U.S. Cl. CPC C12P 7/10
USPC 435/160; 435/165; 210/644

(57) ABSTRACT

A method of removing a fermentation inhibitor from an aqueous mixture of hydrolysis products from cellulosic biomass is disclosed. The method includes providing an aqueous mixture of hydrolysis products from cellulosic biomass, the hydrolysis products comprising at least one of pentose or hexose sugars and a fermentation inhibitor that inhibits a microorganism otherwise capable of fermenting pentose or hexose sugars; and at least partially extracting the fermentation inhibitor from the aqueous mixture with a first extractant by a first liquid-liquid extraction through a first porous membrane to provide a first extract and a raffmate, the first extractant having a water solubility of less than one percent by weight. In some embodiments, the aqueous mixture contains an insufficient amount of the microorganism to ferment the aqueous mixture. In some embodiments, the aqueous mixture is a fermentation broth. A membrane solvent extraction system for carrying out the method is also disclosed.

### CROSS-REFERENCE TO RELATED APPLICATION

This application claims priority to U.S. Provisional Application No. 61/354,506, filed Jun. 14, 2010, the disclosure of which is incorporated by reference in its entirety herein.

### BACKGROUND

Cellulosic biomass materials are generated in large quantities as agricultural waste (e.g., from crops and grasses), wood, waste wood (e.g., from paper mills, logging residues, dead wood, forest brush clearing, orchards, and vineyards), and other wastes (e.g., municipal wastes). Enabling fuel production from cellulosic biomass is becoming increasingly important in efforts to develop sustainable energy without impacting food and feed biomass such as corn.

While significant progress has been made in the conversion of cellulosic biomass to biofuel, it has not been commercialized on a large scale because of technical and economic challenges. One of these challenges is that harsh processes applied during the pre-treatment of cellulosic biomass (e.g., acidic, high temperature steam treatment) create compounds that inhibit fermentation. The presence of these compounds is thought to contribute to low yield in the production of ethanol, for example, from cellulosic biomass. A variety of methods (e.g., evaporation, solvent extraction, overliming, activated charcoal, ion exchange resins, and enzymatic detoxification) have been proposed and tested to remove such inhibitory compounds before fermentation. So far, none of these methods has resulted in an economically viable process for large scale cellulosic biofuel production.

### SUMMARY

There is a continuing need for effective and efficient methods for obtaining fuels from cellulosic biomass. The present disclosure provides a method of removing fermentation inhibitors found in aqueous mixtures of hydrolysis products from cellulosic biomass, and, in some embodiments, producing alcohol. The method includes liquid-liquid extraction through a porous membrane using an extractant that has a solubility in water of less than one percent by weight, that is, less than one gram per 100 milliliters. The porous membrane-solvent extraction stabilizes the interface between the aqueous mixture and the extractant, thereby minimizing emulsification. The low solubility of the extractant minimizes loss of the extractant into the aqueous mixture, thereby maximizing process efficiency and minimizing potential toxification of the raffinate toward fermentation microorganisms. In some embodiments, certain extractants have been found to be remarkably and unexpectedly effective at removing a variety of inhibitors found in hydrolysis products from cellulosic biomass.

In one aspect, the present disclosure provides a method of removing a fermentation inhibitor from an aqueous mixture of hydrolysis products from cellulosic biomass, the method comprising:

providing the aqueous mixture of hydrolysis products from cellulosic biomass, the hydrolysis products comprising at least one of pentose or hexose sugars and a fermentation inhibitor that inhibits a microorganism otherwise capable of fermenting at least one of pentose or hexose sugars; and

at least partially extracting the fermentation inhibitor from the aqueous mixture with a first extractant by a first liquid-liquid extraction through a first porous membrane to provide a first extract and a raffinate, the first extractant having a water solubility of less than one percent by weight,

wherein the raffinate has a lower concentration of the fermentation inhibitor than the aqueous mixture.

In another aspect, the present disclosure provides a method for producing an alcohol from an aqueous mixture of hydrolysis products from cellulosic biomass, the method comprising:

providing the aqueous mixture of hydrolysis products from cellulosic biomass, the hydrolysis products comprising at least one of pentose or hexose sugars and a fermentation inhibitor that inhibits a microorganism otherwise capable of fermenting at least one of pentose or hexose sugars;

at least partially extracting the fermentation inhibitor from the aqueous mixture with a first extractant by a first liquid-liquid extraction through a first porous membrane to provide a first extract and a raffinate, the first extractant having a water solubility of less than one percent by weight, wherein the raffinate has a lower concentration of the fermentation inhibitor than the aqueous mixture;

combining the raffinate with the microorganism; and

fermenting the raffinate to produce the alcohol.

In another aspect, the present disclosure provides a method for producing an alcohol from an aqueous mixture of hydrolysis products from cellulosic biomass, the method comprising:

providing the aqueous mixture of hydrolysis products from cellulosic biomass, the hydrolysis products comprising at least one of pentose or hexose sugars and a fermentation inhibitor that inhibits a microorganism otherwise capable of fermenting at least one of pentose or hexose sugars;

at least partially extracting the fermentation inhibitor from the aqueous mixture with a first extractant by a first liquid-liquid extraction through a first porous membrane to provide a first extract and a raffinate, the first extractant having a water solubility of less than one percent by weight, wherein the raffinate has a lower concentration of the fermentation inhibitor than the aqueous mixture, and wherein a portion of the first extractant becomes entrained in the raffinate;

at least partially extracting the first extractant from the raffinate with a second extractant by a second liquid-liquid extraction to provide an second extract and a fermentable feed solution;

combining the fermentable feed solution with the microorganism; and

fermenting the fermentable feed solution to produce the alcohol.

In another aspect, the present disclosure provides a method for producing an alcohol from an aqueous mixture of hydrolysis products from cellulosic biomass, the method comprising:

fermenting the aqueous mixture to provide a first fermentation broth, the fermentation broth comprising:
a microorganism for fermenting at least one of pentose or hexose sugars;
hydrolysis products from cellulosic biomass, the hydrolysis products comprising at least one of pentose or hexose sugars and a fermentation inhibitor that inhibits the microorganism; and
the alcohol;

at least partially extracting the fermentation inhibitor from the first fermentation broth with a first extractant by a first liquid-liquid extraction through a first porous membrane to provide a first extract and a second fermentation broth, the first extractant having a water solubility of less than one percent by weight,

wherein the second fermentation broth has a lower concentration of the fermentation inhibitor than the first fermentation broth.

In another aspect, the present disclosure provides a membrane solvent extraction system comprising:

a first vessel containing a volume of an aqueous mixture, the aqueous mixture comprising at least one of pentose or hexose sugars and a dissolved fermentation inhibitor that inhibits a microorganism otherwise capable of fermenting pentose or hexose sugars;

a second vessel containing a volume of a first extractant having a water solubility of less than one percent by weight;

a liquid-liquid extraction element comprising:
a plurality of first layer pairs in fluid communication with the first solution vessel, each first layer pair comprising:
   a first polymeric microporous membrane; and
   a first flow channel layer oriented in a first flow direction having a first fluid inlet and a first fluid outlet disposed on first opposing sides of the extraction element; and
a plurality of second layer pairs in fluid communication with the second vessel, with at least one second layer pair being disposed between two first layer pairs and at least one first layer pair being disposed between two second layer pairs so as to form a stack of layers, each second layer pair comprising:
   a second polymeric microporous membrane; and
   a second flow channel layer oriented in a second flow direction different than the first flow direction and having a second fluid inlet and a second fluid outlet disposed on second opposing sides of the extraction element;

wherein at least one of the first or second microporous membranes is disposed between the first flow channel layer and the second flow channel layer of each adjacent first layer pair and second layer pair, and wherein the fermentation inhibitor in the aqueous mixture can transfer into the first extractant across at least the first and second microporous membranes.

In this application, terms such as “a”, “an” and “the” are not intended to refer to only a singular entity, but include the general class of which a specific example may be used for illustration. The terms “a”, “an”, and “the” are used interchangeably with the term “at least one”. The phrases “at least one of” and “comprises at least one of” followed by a list refers to any one of the items in the list and any combination of two or more items in the list. All numerical ranges are inclusive of their endpoints and non-integral values between the endpoints unless otherwise stated.

The terms “first” and “second” are used in this disclosure. It will be understood that, unless otherwise noted, those terms are used in their relative sense only. In particular, in some embodiments certain components may be present in interchangeable and/or identical multiples (e.g., pairs). For these components, the designation of “first” and “second” may be applied to the components merely as a matter of convenience in the description of one or more of the embodiments.

The term “aqueous” refers to comprising water.

The “extractant”, including the first extractant, second extractant, or third extractant, includes one compound or a mixture of compounds. Typically the extractant refers to an organic solvent or a mixture of organic solvents.

The term “raffinate” refers to the portion of the original aqueous mixture that remains after certain components (e.g., at least a portion of the fermentation inhibitor(s)) have been removed by the extractant.

“Liquid-liquid extraction” is a method for transferring a solute dissolved in a first liquid to a second liquid.

The term “entrained” includes when the first extractant is suspended, trapped, or dissolved in the aqueous mixture.

### BRIEF DESCRIPTION OF THE DRAWING

The disclosure may be more completely understood in consideration of the following detailed description of various embodiments in connection with the accompanying drawings, in which:

FIG. 1 is a schematic flow diagram of one illustrative embodiment of the method according to the present disclosure;

FIG. 2 is a schematic flow diagram of another illustrative embodiment of the method according to the present disclosure; and

FIG. 3 is a schematic perspective view of an illustrative membrane extraction module useful for practicing the methods disclosed herein.

### DETAILED DESCRIPTION

The methods according to the present disclosure are useful, for example, for treating an aqueous mixture of hydrolysis products from cellulosic biomass. Exemplary sources of cellulosic biomass include waste wood or bark, waste tree trunk chips from pulp or paper mills, forest waste (e.g., roots, branches, and foliage), orchard and vineyard trimmings, stalks and leaves (i.e., stover) from cotton plants, bamboo, rice, wheat, and corn, waste agricultural products (e.g., rice, wheat, and corn), agricultural byproducts (e.g., bagasse and hemp), and waste paper (e.g., newspaper, computer paper, and cardboard boxes). In some embodiments, the source of cellulosic biomass is corn stover. Some of these materials (e.g., softwood and hardwood materials and crops) are lignocellulosic materials that contain lignin, cellulose, and hemicellulose.

The aqueous mixture of hydrolysis products from cellulosic biomass may be obtained using a variety of pre-treatment methods typically employed to render carbohydrates in cellulosic biomass accessible for fermentation into alcohols (e.g., ethanol or butanol). In some embodiments of any of the methods disclosed herein, the method further comprises hydrolyzing cellulosic biomass. Exemplary pre-treatment methods useful for providing the aqueous mixture of hydrolysis products from cellulosic biomass include acid hydrolysis, high-temperature steam treatment, steam explosion, ammonia freeze explosion, and wet oxidation. High-temperature steam treatment, steam explosion, and wet oxidation can optionally be carried out in the presence of catalytic acid (e.g., sulfuric acid or nitric acid) or base (e.g., sodium hydroxide, sodium carbonate, or ammonia). The pre-treatments can be carried out at temperatures in a range, for example, of 120° C. to 250° C. Typically, during pre-treatment, lignin and hemicellulose are dissolved and/or decomposed in the aqueous phase, and cellulose remains as a solid fraction. In some embodiments, the hydrolysis products from cellulosic biomass are produced by acidic, high-temperature (e.g., 120° C. to 200° C.) steam treatment of corn stover.

An aqueous mixture of hydrolysis products from cellulosic biomass useful for practicing the present disclosure typically contains decomposed hemicellulose, which may contain various pentose and/or hexose sugars (e.g., xylose, arabinose, mannose, galactose, and glucose). In some embodiments, the aqueous mixture of hydrolysis products comprises pentose sugars (in some embodiments, predominantly xylose). In some of these embodiments, the aqueous mixture optionally comprises at least one hexose sugar (e.g., glucose).

The generally harsh conditions employed in the various pre-treatment methods described above typically produce compounds from the cellulosic biomass that are toxic to microorganisms useful for fermentation of pentose and hexose sugars. These compounds are herein termed “fermentation inhibitors” and typically include weak organic acids (e.g., acetic acid, formic acid, and lactic acid); furan derivatives (e.g., furfural and 5-hydroxymethyl-furfural (HMF)); and phenolics (e.g., vanillin). The identity and concentration of the various fermentation inhibitors that can be found in the aqueous mixture of hydrolysis products from cellulosic biomass depends on the particular pre-treatment method described above and the particular source of cellulosic biomass. In some embodiments, the hydrolysis products comprise at least one of acetic acid, lactic acid, formic acid, furfural, 5-hydroxymethylfurfural, or vanillin as the fermentation inhibitor.

Some embodiments of the methods disclosed herein include at least partially extracting the fermentation inhibitor from the aqueous mixture of hydrolysis products from cellulosic biomass with a first extractant by a first liquid-liquid extraction through a first porous membrane to provide a first extract and a raffinate. In some of these embodiments, the aqueous mixture comprises an insufficient amount of the microorganism to ferment the aqueous mixture. For example, the aqueous mixture may contain the microorganism at a concentration of up to 1000 microorganisms per milliliter (mL). In some embodiments, the aqueous mixture may contain the microorganism at a concentration of up to 800/mL, 600/mL, or less. In some embodiments, the aqueous mixture is free of the microorganism. In some embodiments, the aqueous mixture is not a fermentation broth and/or is not fermentable. Similarly, in some of these embodiments, the raffinate comprises an insufficient amount of the microorganism to ferment the raffinate. For example, the raffinate may contain the microorganism at a concentration of up to 1000/mL, 800/mL, 600/mL, or less. In some embodiments, the raffinate is free of the microorganism. In some embodiments, the raffinate is not a fermentation broth and/or is not fermentable. In some of these embodiments, the method can be useful as a detoxification procedure before the fermentation of the raffinate. Accordingly, in some embodiments of methods of producing an alcohol from an aqueous solution of hydrolysis products from cellulosic biomass, the method comprises combining the raffinate with the microorganism and fermenting the raffinate to produce an alcohol.

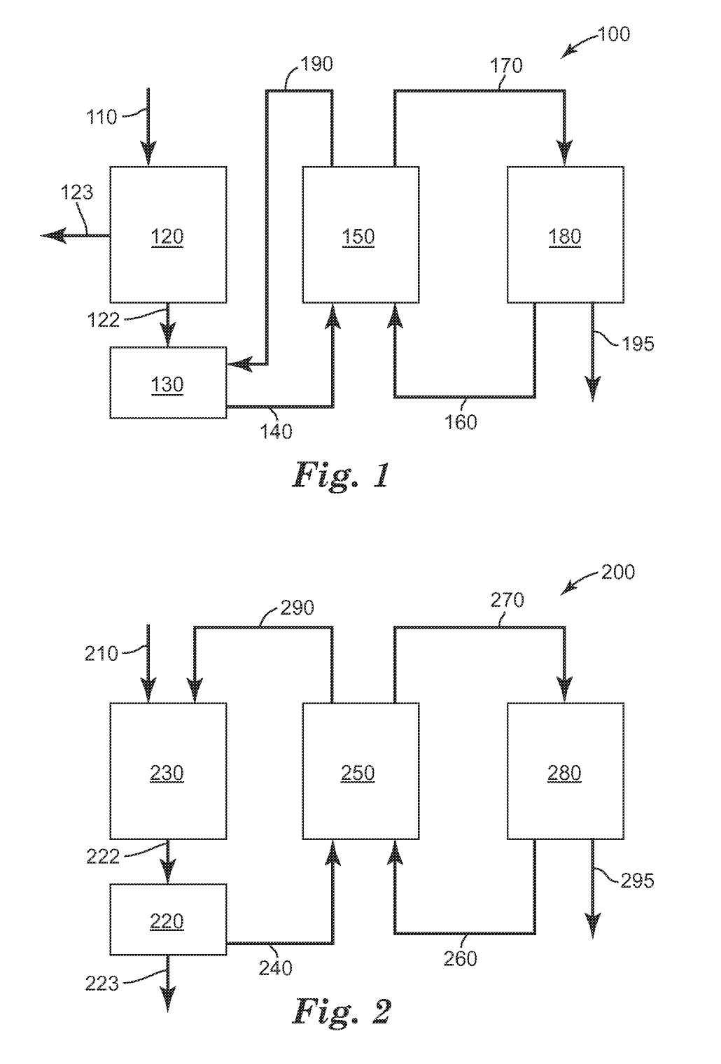

A flow diagram 100 of an exemplary embodiment of the method according to the present disclosure is shown in FIG. 1. In flow diagram 100, aqueous mixture 110, which contains hydrolysis products from cellulosic biomass including at least one of pentose or hexose sugars and a fermentation inhibitor, is placed into extractor 120 along with the first extractant. In extractor 120, the aqueous mixture 110 and the first extractant are brought into intimate contact with each other such that fermentation inhibitors partition between the two. First extract 123 is typically removed and can be optionally separated to recover the first extractant and the fermentation inhibitors. For example, the first extract 123 may be subjected to a liquid-liquid extraction (e.g., through a porous membrane) with water or other aqueous solution to extract the inhibitors from the first extractant. The first extractant can then optionally be reused (e.g., by adding back to extractor 120). In the illustrated embodiment, raffinate 122 is then introduced into fermenter 130 along with microorganisms for carrying out the fermentation. Fermentation broth 140 can then be transported in some embodiments to extractor 150. In extractor 150, fermentation broth 140 and a third extractant are brought into intimate contact with each other such that the alcohol produced partitions between fermentation broth 140 and the third extractant. Third extract 170, which contains third extractant 160 and the produced alcohol, is then transported to recovery unit 180 where the alcohol 195, optionally mixed with water, is removed from third extract 170 (e.g., by vacuum distillation) such that third extractant 160 is regenerated and recycled into extractor 150. Likewise, extracted fermentation broth 190 can be returned to fermenter 130, which can be periodically replenished with additional raffinate 122 as desired.

In other embodiments, the present disclosure provides methods of removing fermentation inhibitors directly from a fermentation broth. In these embodiments, the aqueous mixture is a fermentation broth, and the method includes include at least partially extracting the fermentation inhibitor from the first fermentation broth with a first extractant by a first liquid-liquid extraction through a first porous membrane to provide a first extract and a second fermentation broth. In these embodiments, the aqueous mixture includes a microorganism in an amount typically sufficient (e.g., in the absence of inhibitors) to ferment at least one of pentose or hexose sugars. For example, the concentration of the microorganism may be more than 1000, 1200, 1500, or 2000 microorganisms per mL. A flow diagram of one exemplary embodiment for removing fermentation inhibitors directly from a fermentation broth is shown in FIG. 2. In flow diagram 200, aqueous mixture 210, which contains a microorganism for fermenting at least one of pentose or hexose sugars and hydrolysis products from cellulosic biomass including at least one of pentose or hexose sugars and a fermentation inhibitor, is placed into fermenter 230 and allowed to form a fermentation broth 222. Fermentation broth 222 can be transported to a first extractor 220, in which the aqueous mixture 210 and the first extractant are brought into intimate contact with each other such that fermentation inhibitors partition between the two. First extract 223 is typically removed and can be optionally separated to recover the first extractant, the fermentation inhibitors, and any alcohol produced. For example, the first extract 223 may be subjected to a liquid-liquid extraction (e.g., through a porous membrane) with water or other aqueous solution to extract the inhibitors from the first extractant. The first extractant can then optionally be reused (e.g., by adding back to extractor 220). The second fermentation broth 240 from which fermentation inhibitors have been at least partially removed may be returned to the fermenter 230, or as shown in the illustrated embodiment, can be transported to second extractor 250. In extractor 250, second fermentation broth 240 and a third extractant are brought into intimate contact with each other such that the produced alcohol partitions between second fermentation broth 240 and the third extractant. Third extract 270, which contains third extractant 260 and the produced alcohol, is then transported to recovery unit 280 where the produced alcohol 295, optionally mixed with water, is removed from third extract 270 (e.g., by vacuum distillation) such that third extractant 260 is regenerated and recycled into extractor 250. Likewise, extracted second fermentation broth 290 is returned to fermenter 230, which is periodically replenished with additional aqueous mixture 210 as necessary to replace components that have been removed during the process. Optionally, the first extractor 220 and second extractor 250 can be combined into one extractor to remove both the produced alcohol and the fermentation inhibitors in one step, depending on the selection of the solvent.

Typically, fermentation broths useful for practicing the present disclosure may be prepared by combining the aqueous mixture or the raffinate and a microorganism in a vessel (e.g., fermenter, vat), and maintaining the mixture at a temperature at which fermentation can occur (e.g., in a range of from 15° C. to 45° C.). Fermenters are widely commercially available and are described, for example, in U.S. Pat. No. 4,298,693 (Wallace).

The microorganism that can ferment at least one of pentose or hexose sugars in any of the above embodiments may be, for example, one of various strains of thermophilic bacteria, Zymomonas bacteria, Escherichia coli, or brewer's yeast. In some embodiments, the hydrolysis products comprise pentose sugars, and the microorganism is a pentose-fermenting microorganism (e.g., Zymomonas bacteria or Escherichia coli). In some of these embodiments, the microorganism can also ferment at least one hexose sugar.

In some embodiments of the methods disclosed herein, including the methods described above in connection with FIGS. 1 and 2, the method further comprises recovering at least a portion of the alcohol (e.g., ethanol or butanol). As described above, the alcohol can be extracted by a third liquid-liquid extraction with a third extractant from a second extractor 150 or 250. Exemplary third extractants include decyl alcohol, 2,6-dimethyl-4-heptanol, dodecane, and combinations thereof. Exemplary third extractants also include those described in U.S. Pat. No. 7,122,709 (Fanselow et al.). The alcohol can be removed from the third extractant, for example, by vacuum distillation.

The first extractant useful for practicing any of the methods disclosed herein has a solubility in water of up to one percent by weight. In some embodiments, the first extractant has a solubility in water of up to 0.8, 0.6, 0.5, 0.4, or 0.35 percent by weight. As described above, the first extractant may be a single compound or a combination of compounds. When the first extractant is a combination of compounds, in some embodiments, each of the compounds has a solubility in water of up to 0.8, 0.6, 0.5, 0.4, or 0.35 percent by weight. First extractants with a solubility in water of up to one percent by weight will tend not to be dissolved, entrained, or otherwise lost in the aqueous mixture or dissolved, entrained, or otherwise lost to only a small extent. The benefit of low extractant loss in the methods disclosed herein is better economic viability of the methods and, depending on the toxicity of the extractant, minimization of the effects of extractant toxicity on the microorganism. Water solubilities can be obtained from the literature, for example, from IUPAC-NIST Solubility Data Series, Version 1, NIST Standard Reference Database 106, updated March 2006 available online at http://srdata.nist.gov/solubility/index.aspx; Lange's Handbook of Chemistry (16th edition), ed. Speight, James G, McGraw-Hill, NY, 2005; and Yaws' Handbook of Properties for Environmental and Green Engineering, ed. Yaws, Carl L, McGraw-Hill, NY, 2008. In case of a conflict between data sources, the water solubility for a particular extractant will be that reported in the IUPAC-NIST Solubility Data Series.

Some solvent extraction techniques that have been reported as detoxification techniques for fermentation of cellulosic materials utilize ethyl acetate, diethyl ether, or 1-butanol, which have water solubilities of 8.3, 6.9, 7.5 grams per 100 mL (8.3, 6.9, and 7.5 percent), respectively. The data for ethyl acetate and 1-butanol were obtained from the IUPAC-NIST Solubility Data Series, and solubility in water for diethyl ether was obtained from Yaws' Handbook of Properties for Environmental and Green Engineering. These solubilities in water can lead to the problems of solvent loss and toxicity discussed above.

In some embodiments, the first extractant useful for practicing any of the methods disclosed herein has a solubility parameter in a range from 7 to 14 (calories per cubic centimeter)^{1/2}, in some embodiments, 7.5 to 11 (calories per cubic centimeter)^{1/2}. In some embodiments, the first extractant has a solubility parameter of up to 14 (calories per cubic centimeter)^{1/2}. High solubility parameter values that approach the value of water (23.4 (calories per cubic centimeter)^{1/2}) tend to lead to a water solubility of more than one percent by weight, which can lead to the problems of extractant loss in the method and toxicity discussed above. In some embodiments, the first extractant has a solubility parameter of at least 7 (calories per cubic centimeter)^{1/2}. Extractants with solubility parameters of less than 7 (calories per cubic centimeter)^{1/2} typically have very low polarity and may, in some circumstances, not as effectively remove fermentation inhibitors from aqueous mixtures. In some embodiments, the first extractant has a solubility parameter of up to 13.5, 13, 12.5, 12, 11.5, 11, 10.5, 10, or 9.5 (calories per cubic centimeter)^{1/2}. In some embodiments, the first extractant has a solubility parameter of at least 7.5, 7.6, 7.7, 7.8, 7.9, 8, 8.1, 8.2, 8.3, 8.4, 8.5, or 8.6 (calories per cubic centimeter)^{1/2}. As described above, the first extractant may be a single compound or a combination of compounds. When the first extractant is a combination of compounds, in some embodiments, each of the compounds has a solubility parameter in a range from 7 to 14, 7.5 to 10.5, up to 14, or at least 7 or 7.5. The solubility parameter of an extractant described herein is calculated by computer simulation using computational chemistry molecular dynamics simulations (CERIUS 2/OFF, Accelrys, San Diego, Calif.) employing the Dreiding Force Field. Solubility parameters are computed from the cohesive energy density after molecular dynamics simulations on periodic bulk samples of each solvent. The solubility parameters for a number of exemplary first extractants are reported in the Examples, below.

In some embodiments, the first extractant useful for practicing any of the methods disclosed herein has a solubility parameter in a range from 8.6 to 11 (calories per cubic centimeter)^{1/2}. As described above, the first extractant may be a single compound or a combination of compounds. When the first extractant is a combination of compounds, in some embodiments, each of the compounds has a solubility parameter in this range. As shown in the Examples, below, extractants with a solubility parameter in the range of 8.6 to 11 typically, and unexpectedly, have remarkably high yields for extracting a variety of fermentation inhibitors (e.g., acetic acid, HMF, furfural, vanillin, and combinations thereof) and low yields for extracting pentose and/or hexose sugars (e.g., xylose or glucose).

In some embodiments, the first extractant useful for practicing any of the methods disclosed herein extracts only minimal amounts of the at least one of pentose or hexose sugars from the aqueous mixture. In some embodiments, the solubility of the at least one of pentose or hexose sugars in the first extractant is up to one (in some embodiments, up to 0.9, 0.8, 0.7, 0.6, or 0.5) percent by weight. As described above, the first extractant may be a single compound or a combination of compounds. When the first extractant is a combination of compounds, in some embodiments, the solubility of the at least one of pentose or hexose sugars in the first extractant is up to one (in some embodiments, up to 0.9, 0.8, 0.7, 0.6, or 0.5) percent by weight in each of the compounds. Minimal extraction of the at least one of pentose or hexose sugars (e.g., xylose or glucose) is desirable so that fermentable sugars are not lost in the first extraction process, which would lower the yield of an alcohol produced by fermentation of the sugars.

In some embodiments, the first extractant comprises a straight-chain or branched alcohol having from 8 to 20 (in some embodiments 8 to 16, 8 to 14, or 8 to 12) carbon atoms. In some of these embodiments, the straight-chain or branched alcohol is a primary alcohol. In some embodiments, the first extractant comprises a straight-chain alcohol having from 8 to 20 (in some embodiments 8 to 16, 8 to 14, or 8 to 12) carbon atoms. In some of these embodiments, the first extractant comprises at least one of 2-ethyl-1-hexanol, 1-octanol, 2-octanol, 1-decanol, or 1-dodecanol. In some of these embodiments, the first extractant comprises at least one of 1-octanol, 2-octanol, 1-decanol, or 1-dodecanol.

In some embodiments, the first extractant comprises at least one of acetophenone, 3-nonanone, ethyl butyrate, dibutyl maleate, 2-methyl-2-pentenal, or benzaldehyde. In some embodiments, the first extractant is other than or does not comprise supercritical carbon dioxide.

In some embodiments of the methods according to the present disclosure, depending on the choice of materials and conditions, a portion of the first extractant becomes entrained in the raffinate. In some of these embodiments, the first extractant becomes entrained in the raffinate at a level that inhibits or kills the microorganism. In some embodiments, the method further comprises at least partially extracting the entrained first extractant from the raffinate with a second extractant by a second liquid-liquid extraction to provide a second extract and a fermentable feed solution. In some embodiments of the methods according to the present disclosure, a portion of the first extractant becomes entrained in the second fermentation broth. In some of these embodiments, the first extractant becomes entrained in the second fermentation broth at a level that inhibits or kills the microorganism. In some of these embodiments, the method further comprises at least partially extracting the entrained first extractant from the second fermentation broth with a second extractant by a second liquid-liquid extraction. In some embodiments of methods that employ a second liquid-liquid extraction, the second liquid-liquid extraction is carried out through a second porous membrane. In some embodiments, the second extractant is an alkane or a combination of alkanes. In some of these embodiments, the alkane has 8 to 16 (in some embodiments, 8 to 14 or 8 to 12) carbon atoms and be straight-chained, branched, or cyclic. In some embodiments, the second extractant comprises at least one of n-octane, n-nonane, n-decane, n-undecane, n-dodecane, n-tetradecane, n-hexadecane, 2-methylnonane, 4-ethyl-2-methyloctane, 2,2-dimethyldecane, 4-methyldecane, 2,6-dimethyldecane, 1,2,4-trimethylcyclohexane, cis- or trans-decalin.

In some embodiments wherein a portion of the first extractant becomes entrained in the raffinate and the method further comprises at least partially extracting the entrained first extractant from the raffinate with a second extractant by a second liquid-liquid extraction to provide a second extract and a fermentable feed solution, the method further comprises combining the fermentable feed solution with the microorganism; and fermenting the fermentable feed solution to produce an alcohol (e.g., ethanol or butanol). The fermentation conditions and microorganisms described above may also be useful for these embodiments.

A variety of porous membranes and membrane extraction apparatuses may be useful for practicing the present disclosure. The following embodiments of porous membranes and apparatuses may be useful for the first liquid-liquid extraction (that is, the extraction of the fermentation inhibitors), the second liquid-liquid extraction (that is, the extraction of the entrained first extractant), or the third liquid-liquid extraction (that is, the extraction of the alcohol) described herein. The membrane extraction apparatus may be of any design as long as the extractant (e.g., first, second, or third extractant) and aqueous solution to be extracted (e.g. the aqueous mixture, raffinate, or fermentation broth) have a liquid-liquid interface within at least one pore, typically a plurality of pores, of the porous membrane. In general, the rate of extraction depends on the area of the liquid-liquid interface. Thus, membrane extraction apparatus designs that have large membrane surface areas are typically desirable, although designs having relatively smaller membrane surface areas may also be useful.

To facilitate formation of an interface between the aqueous solution and the extractant within the porous membrane, whichever of the aqueous solution or the extractant wets the membrane least well may be maintained at higher pressure than the other. For example, in the case of a hydrophobic porous membrane the aqueous solution may have a higher fluid pressure than the extractant. This pressure differential should typically be sufficient to substantially immobilize the interface between the aqueous solution and extractant, but preferably not large enough to cause damage to the porous membrane. The pressure differential may be achieved by a variety of known methods including a restriction valve (e.g., a back-pressure valve on an extract outlet port), a fluid height differential, or the like. If present, the pressure differential between the aqueous solution and the extractant may be, for example, at least 10 cm water at 4° C. (1 kPa), at least 1 PSI (6.9 kPa), and may be up to 13 PSI (90 kPa), although higher and lower pressures may also be used.

In some embodiments of the methods disclosed herein, the first porous membrane is a microporous membrane. In some embodiments, the second porous membrane is a microporous membrane. Microporous membranes useful for practicing the present disclosure typically have micrometer-sized pores (that is, micropores) that extend between major surfaces of the membrane. The micropores may be, for example, isolated or interconnected. The microporous membrane may be formed from any material having micropores therethrough, for example, a microporous thermoplastic polymer. The microporous membrane may, for example, be flexible or rigid. In some embodiments according to the present invention, useful thermoplastic microporous membranes may comprise a blend of similar or dissimilar thermoplastic polymers, each optionally having a different molecular weight distribution (e.g., a blend of ultrahigh molecular weight polyethylene and high molecular weight polyethylene).

Micropore size, thickness, and composition of the microporous membranes typically determine the rate of extraction in the methods disclosed herein. The size of the micropores of the microporous membrane should be sufficiently large to permit contact between the aqueous solution and the extractant within the micropores (e.g., to form a liquid-liquid extraction interface), but not so large that flooding of the aqueous solution through the microporous membrane into the extractant occurs.

Microporous membranes useful for practice of the present invention may be, for example, hydrophilic or hydrophobic. Microporous membranes can be prepared by methods well known in the art and are described in, for example, U.S. Pat. Nos. 3,801,404 (Druin et al.); 3,839,516 (Williams et al.); 3,843,761 (Bierenbaum et al.); 4,255,376 (Soehngen et al.); 4,257,997 (Soehngen et al.); 4,276,179 (Soehngen); 4,973,434 (Sirkar et al.), and/or are widely commercially available from suppliers such as, for example, Celgard, Inc. (Charlotte, N.C.); Tetratec, Inc. (Ivyland, Pa.); Nadir Filtration GmbH (Wiesbaden, Germany); or Membrana, GmbH (Wuppertal, Germany). Exemplary hydrophilic membranes include membranes of microporous polyamide (e.g., microporous nylon), microporous polycarbonate, microporous ethylene vinyl alcohol copolymer, and microporous hydrophilic polypropylene. Exemplary hydrophobic membranes include membranes of microporous polyethylene, microporous polypropylene (e.g., thermally induced phase separation microporous polypropylene), and microporous polytetrafluoroethylene.

Typically, the mean pore size of useful microporous membranes (e.g., as measured according to ASTM E1294-89 (1999) “Standard Test Method for Pore Size Characteristics of Membrane Filters Using Automated Liquid Porosimeter”) may be greater than about 0.07 micrometer (e.g., greater than 0.1 micrometer or greater than 0.25 micrometer), and may be less than 1.4 micrometers (e.g., less than 0.4 micrometer or less than 0.3 micrometer), although microporous membranes having larger or smaller mean pore sizes may also be used. In order to reduce emulsion formation and/or flooding across the membrane, the microporous membrane may be substantially free of pores, tears, or other holes that exceed 100 micrometers in diameter.

Useful microporous membranes typically have a porosity in a range of from at least about 20 percent (e.g., at least 30 percent or at least 40 percent) up to 80 percent, 87 percent, or even 95 percent, based on the volume of the microporous membrane. Typically, useful microporous membranes have a thickness of at least about 25 micrometers (e.g., at least 35 micrometers or at least 40 micrometers), and/or may have a thickness of less than about 80 micrometers (e.g., less than 60 micrometers or even less than 50 micrometers), although membranes of any thickness may be used. Typically, microporous membranes should be mechanically strong enough, alone or in combination with an optional porous support member, to withstand any pressure difference that may be imposed across the microporous membrane under the intended operating conditions.

Multiple microporous membranes may be used in series or in parallel for any of the liquid-liquid extractions disclosed herein. Exemplary membrane forms include sheets, bags, and tubes and may be substantially planar or nonplanar (e.g., pleated, spiral wound cartridge, plate-frame, or hollow fiber bundle). In some embodiments of methods disclosed herein, a microporous membrane may comprise a microporous hollow fiber membrane as described in, for example, U.S. Pat. Nos. 4,055,696 (Kamada et al.); 4,405,688 (Lowery et al.); 5,449,457 (Prasad). Of course, the nature of the extraction apparatus (e.g., shape, size, components) may vary depending on the form of the membrane chosen.

The microporous membrane may comprise at least one hydrophobic (that is, not spontaneously wet out by water) material. Exemplary hydrophobic materials include polyolefins (e.g., polypropylene, polyethylene, polybutylene, copolymers of any of the forgoing and, optionally, an ethylenically unsaturated monomer), and combinations thereof. If the microporous membrane is hydrophobic, a positive pressure may be applied to the aqueous solution relative to the extractant to aid in wetting the microporous membrane.

In some embodiments of the methods disclosed herein, the microporous membrane may be hydrophilic, for example, a hydrophilic microporous polypropylene membrane having a nominal average pore size in a range of from 0.2 to 0.45 micrometers (e.g., as marketed under the trade designation “GH POLYPRO MEMBRANE” by Pall Life Sciences, Inc., Ann Arbor, Mich.). If the microporous membrane is hydrophilic, positive pressure may be applied to the extractant relative to the aqueous solution to facilitate immobilization of the liquid-liquid interface within the membrane. Exemplary membranes include microporous membranes as described in U.S. Pat. Nos. 3,801,404 (Druin et al.); 3,839,516 (Williams et al.); 3,843,761 (Bierenbaum et al.); 4,255,376 (Soehngen); 4,257,997 (Soehngen et al.); and 4,276,179 (Soehngen); 4,726,989 (Mrozinski); 5,120,594 (Mrozinski); and 5,238,623 (Mrozinski).

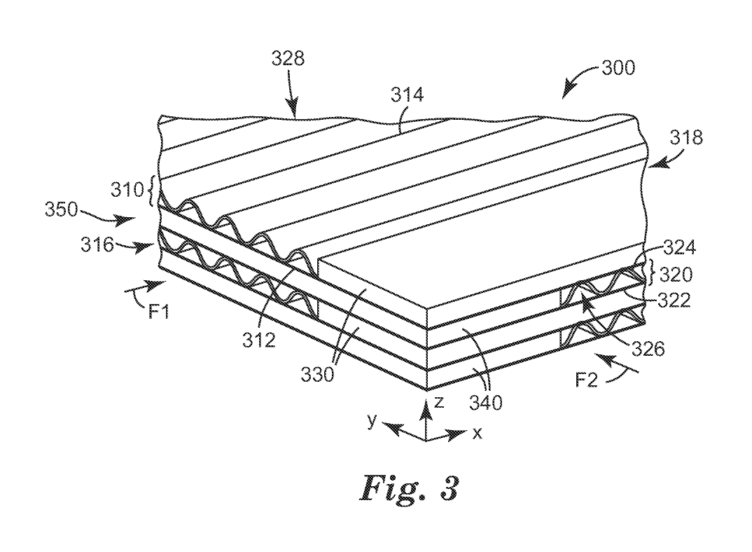

Several useful microporous membrane extraction apparatuses are described, for example, in U.S. Pat. No. 7,105,089 (Fanselow et al.) U.S. Pat. App. Pub. No. 2007/0119771 (Skukar et al.). An exemplary embodiment of a membrane extraction element of a membrane extraction apparatus useful for practicing the present disclosure is shown in FIG. 3. The membrane extraction element 300 includes a first layer pair 310 and a second layer pair 320. The second layer pair 320 is disposed adjacent the first layer pair 310 forming a stack of layers 350. The stack of layers 350 has an x-, y-, and z-axis as shown in FIG. 3. The z-axis is the thickness direction of the stack of layers 350. The x-axis and y-axis are both in-plane axes of the stack of layers 350 and are orthogonal to one another in the illustrated embodiment.

The first layer pair 310 includes first polymeric microporous membrane 312 and a first flow channel layer 314 oriented in a first flow F₁ direction (along the x-axis of FIG. 3) having a fluid inlet 316 and a fluid outlet 318 disposed on first opposing sides of the extraction element 300 (along the y-axis of FIG. 3). Thus, in the illustrative embodiment shown in FIG. 3, the first flow F₁ direction is orthogonal to the first opposing sides of the liquid-liquid extraction element 300.

The second layer pair 320 includes a second polymeric microporous membrane 322 and a second flow channel layer 324 oriented in a second flow direction F₂ (along the y-axis of FIG. 3) different than the first flow direction F₁ and having a fluid inlet 326 and a fluid outlet 328 disposed on second opposing sides (along the x-axis of FIG. 3) of the membrane extraction element 300. Thus, in the illustrative embodiment shown in FIG. 3, the second flow F₂ direction is orthogonal to the second opposing sides of the membrane extraction element 300. The first microporous membrane 312 is shown disposed between the first flow channel layer 314 and the second flow channel layer 324. In the illustrated embodiment, the first flow direction F₁ is orthogonal to the second flow direction F₂, but this is not required.

In many embodiments, the liquid-liquid extraction element 300 includes a plurality (two or more) of alternating first layer pairs 310 and second layer pairs 320. In some embodiments, the membrane extraction element 300 includes from 10 to 2000, or 25 to 1000, or 50 to 500 alternating first layer pairs 310 and second layer pairs 320 stacked in vertical registration (along the z-axis) where the first flow direction F₁ (along the x-axis) is orthogonal to the second flow direction F₂ (along the y-axis).

The flow channel layers 314, 324 and the microporous membrane layers 312, 322 have layer thicknesses (along the z-axis) of any useful value. In many embodiments, the first flow channel layer 314 and the second flow channel layer 324 each has a thickness in a range from 10 to 250, or 25 to 150 micrometers. In many embodiments, the first polymeric microporous membrane 312 and the second polymeric microporous membrane 322 each has a thickness in a range from 1 to 200, or 10 to 100 micrometers. The extraction element 300 has an overall thickness (along the z-axis) of any useful value. In some embodiments, the extraction element 300 has an overall thickness (along the z-axis) in a range from 5 to 100, or 10 to 50 centimeters.

The membrane extraction element 300 can have any useful shape (e.g., a rectilinear shape). The extraction element 300 has a width (along the y-axis) and a length (along the x-axis) of any useful value. In some embodiments, the extraction element 300 has an overall width (along the y-axis) in a range from 10 to 300, or 50 to 250 centimeters. In some embodiments, the extraction element 300 has an overall length (along the x-axis) in a range from 10 to 300, or 50 to 250 centimeters. In one embodiment, the extraction element 300 length is equal or substantially equal to its width.

The first and second flow channel layers 314, 324 can be formed of the same or different material and take the same or different forms, as desired. The first and second flow channel layers 314, 324 can allow liquid to flow between first and second microporous membranes 312, 322. In many embodiments, the first and second flow channel layers 314, 324 can be structured such that the first and second flow channel layers 314, 324 form flow channels between the microporous membranes 312, 322. In some embodiments, the first and second flow channel layers 314, 324 are non-porous and formed of a polymeric material (e.g., a polyolefin).

In some embodiments, the first and second flow channel layers 314, 324 are corrugated (having parallel alternating peaks and valleys) to provide flow channels between the microporous membranes 312, 322. In many embodiments, the corrugations provide flow channels that are parallel the flow direction. These corrugations can have any useful pitch (distance between adjacent peaks or valleys). In some embodiments, the corrugations have a pitch in a range from 0.05 to 1, or from 0.1 to 0.7 centimeter. The corrugations can be formed by any useful method (e.g., embossing or molding).

As shown in FIG. 3, an exemplary configuration of the extraction element 300 includes a first layer pair 310 having first planar polymeric microporous membrane 312 and a first corrugated flow channel layer 314 oriented in a first flow F₁ direction (along the x-axis of FIG. 3). Thus, in the illustrative embodiment shown in FIG. 3, the first flow F₁ direction is parallel to the corrugations of the first corrugated flow channel layer 314. The second layer pair 320 includes a second planar polymeric microporous membrane 322 and a second corrugated flow channel layer 324 oriented in a second flow direction F₂ (along the y-axis of FIG. 3) orthogonal to the first flow direction F₁ and parallel to the corrugations of the second corrugated flow channel layer 324. Thus, in the illustrative embodiment shown, the first flow direction F₁ is orthogonal to the second flow direction F₂, and the corrugations of the first corrugated flow channel layer 314 are orthogonal to the corrugations of the second corrugated flow channel layer 324.

The extraction element 300 can optionally include layer seals 330, 340 disposed along the selected edges of the extraction element 300. First layer seals 330 can be formed between the porous membrane of one layer, and the flow channel layer below it (in the flow direction of that flow channel layer) along opposing sides of the liquid-liquid extraction element 300. Second layer seals 340 can be formed between the porous membrane of one layer, and the flow channel layer below it (in the flow direction of that flow channel layer) along opposing sides of the extraction element 300. In some embodiments, first and second layer seals, 330, 340 alternate on opposing sides, as shown in FIG. 3.

In some embodiments, layer seals 330, 340 between the layers can be beads of adhesive, a sonic seal, or a heat seal. Thus, a two-directional liquid-liquid extraction flow module can be created, in which a first fluid flows through the module in a first direction, passing through the corrugated spacers and porous membrane of every other layer, contacting the porous membrane layers uniformly on one side; and a second fluid is directed to flow through the liquid-liquid extraction module in a second direction (often orthogonal) to the first direction, passing through the corrugated spacers of layers alternate to the first, contacting the membrane layers uniformly on the other side.

In some embodiments, a first porous non-woven layer (not shown) is disposed between the first polymeric microporous membrane 312 and the first flow channel layer 314 and a second porous non-woven layer (not shown) is disposed between the second polymeric microporous membrane 322 and the second flow channel layer 324. This porous non-woven layer can assist in reinforcing the microporous membrane layer and/or the flow channel layer. The porous non-woven layer can be any useful material such as, for example, a spunbond layer. This porous non-woven layer can be optionally attached (adhesive, ultrasonic seal, heat seal, and the like) to the polymeric microporous membrane and/or flow channel layer.

In some embodiments, a first vessel (not shown) containing a volume of an aqueous mixture, the aqueous mixture comprising at least one of pentose or hexose sugars and a dissolved fermentation inhibitor that inhibits a microorganism otherwise capable of fermenting pentose or hexose sugars, is in fluid communication with a plurality of first layer pairs 310. The aqueous mixture may be a fermentation broth or may contain the microorganism in an insufficient amount to ferment the aqueous mixture as described above. In some of these embodiments, a second vessel (not shown) containing a volume of a first extractant (including a first extractant as described in any of the embodiments above) having a water solubility of less than one percent by weight is in fluid communication with a plurality of second layer pairs 320. The first vessel may be connected to a first entrance manifold (not shown) in fluid communication with the first fluid inlet 316 of each first layer pair 310. The aqueous mixture may enter all of the first layer pairs 310 through the manifold. In some embodiments of a membrane extraction system disclosed herein, a first exit manifold, through which a raffinate exits from all of the first layer pairs 310, is in fluid communication with the first fluid outlet 318 of each first layer pair 310. In some embodiments of a membrane extraction system disclosed herein, a second entrance manifold (not shown) in fluid communication with the second fluid inlet 326 of each second layer pair 320 is connected to the second vessel and allows the first extractant to enter all of the second layer pairs 320. In some embodiments, a second exit manifold (not shown), through which an extract exits from all of the second layer pairs 320, is in fluid communication with the second fluid outlet 328 of each second layer pair 320.

The methods according to the present disclosure typically increase the rate of fermentation to produce an alcohol (e.g., ethanol or butanol) and/or increase the yield of alcohol produced in a fermentation process. As shown in the Examples below, when methods according to the present disclosure are used to at least partially remove fermentation inhibitors from aqueous mixtures containing hydrolysis products from cellulosic biomass, production of ethanol is observed at a much higher level than when fermentation is carried out on aqueous mixtures from cellulosic biomass not subjected to extraction of fermentation inhibitors. The methods disclosed herein are advantageously effective at removing more than one fermentation inhibitor at a time (e.g., at least 2, 3, or 5 fermentation inhibitors).

### Selected Embodiments of the Disclosure

In a first embodiment, the present disclosure provides a method of removing a fermentation inhibitor from an aqueous mixture of hydrolysis products from cellulosic biomass, the method comprising:

providing the aqueous mixture of hydrolysis products from cellulosic biomass, the hydrolysis products comprising at least one of pentose or hexose sugars and a fermentation inhibitor that inhibits a microorganism otherwise capable of fermenting at least one of pentose or hexose sugars; and

at least partially extracting the fermentation inhibitor from the aqueous mixture with a first extractant by a first liquid-liquid extraction through a first porous membrane to provide a first extract and a raffinate, the first extractant having a water solubility of less than one percent by weight,

wherein the raffinate has a lower concentration of the fermentation inhibitor than the aqueous mixture.

In a second embodiment, the present disclosure provides the method of the first embodiment, wherein the method further is a method for producing an alcohol, the method further comprising:

combining the raffinate with the microorganism; and

fermenting the raffinate to produce the alcohol.

In a third embodiment, the present disclosure provides the method of the first embodiment, wherein a portion of the first extractant becomes entrained in the raffinate, the method further comprising:

at least partially extracting the entrained first extractant from the raffinate with a second extractant by a second liquid-liquid extraction to provide an second extract and a fermentable feed solution.

In a fourth embodiment, the present disclosure provides the method of the third embodiment, wherein the method further is a method for producing an alcohol, the method further comprising:

combining the fermentable feed solution with the microorganism; and

fermenting the fermentable feed solution to produce the alcohol.

In a fifth embodiment, the present disclosure provides the method of any of the first to fourth embodiments, the method further comprising:

at least partially extracting the fermentation inhibitor from the first extract with an aqueous solution by a fourth liquid-liquid extraction.

In a sixth embodiment, the present disclosure provides the method of the fifth embodiment, wherein the fourth liquid-liquid extraction is carried out through a fourth porous membrane.

In a seventh embodiment, the present disclosure provides a method for producing an alcohol from an aqueous mixture of hydrolysis products from cellulosic biomass, the method comprising:

providing the aqueous mixture of hydrolysis products from cellulosic biomass, the hydrolysis products comprising at least one of pentose or hexose sugars and a fermentation inhibitor that inhibits a microorganism otherwise capable of fermenting at least one of pentose or hexose sugars;

at least partially extracting the fermentation inhibitor from the aqueous mixture with a first extractant by a first liquid-liquid extraction through a first porous membrane to provide a first extract and a raffinate, the first extractant having a water solubility of less than one percent by weight, wherein the raffinate has a lower concentration of the fermentation inhibitor than the aqueous mixture;

combining the raffinate with the microorganism; and

fermenting the raffinate to produce the alcohol.

In an eighth embodiment, the present disclosure provides a method for producing an alcohol from an aqueous mixture of hydrolysis products from cellulosic biomass, the method comprising:

providing the aqueous mixture of hydrolysis products from cellulosic biomass, the hydrolysis products comprising at least one of pentose or hexose sugars and a fermentation inhibitor that inhibits a microorganism otherwise capable of fermenting at least one of pentose or hexose sugars;

at least partially extracting the fermentation inhibitor from the aqueous mixture with a first extractant by a first liquid-liquid extraction through a first porous membrane to provide a first extract and a raffinate, the first extractant having a water solubility of less than one percent by weight, wherein the raffinate has a lower concentration of the fermentation inhibitor than the aqueous mixture, and wherein a portion of the first extractant becomes entrained in the raffinate;

at least partially extracting the entrained first extractant from the raffinate with a second extractant by a second liquid-liquid extraction to provide an second extract and a fermentable feed solution;

combining the fermentable feed solution with the microorganism; and

fermenting the fermentable feed solution to produce the alcohol.

In a ninth embodiment, the present disclosure provides the method of the seventh or eighth embodiment, the method further comprising:

at least partially extracting the fermentation inhibitor from the first extract with an aqueous solution by a fourth liquid-liquid extraction.

In a tenth embodiment, the present disclosure provides the method of embodiment 9, wherein the fourth liquid-liquid extraction is carried out through a fourth porous membrane.

In an eleventh embodiment, the present disclosure provides the method of any one of embodiments 1 to 10, wherein the aqueous mixture comprises an insufficient amount of the microorganism to ferment the aqueous mixture.

In a twelfth embodiment, the present disclosure provides a method for producing an alcohol from an aqueous mixture of hydrolysis products from cellulosic biomass, the method comprising:

fermenting the aqueous mixture to provide a first fermentation broth, the fermentation broth comprising:
a microorganism for fermenting at least one of pentose or hexose sugars;
hydrolysis products from cellulosic biomass, the hydrolysis products comprising at least one of pentose or hexose sugars and a fermentation inhibitor that inhibits the microorganism; and
   the alcohol;

at least partially extracting the fermentation inhibitor from the first fermentation broth with a first extractant by a first liquid-liquid extraction through a first porous membrane to provide a first extract and a second fermentation broth, the first extractant having a water solubility of less than one percent by weight,

wherein the second fermentation broth has a lower concentration of the fermentation inhibitor than the first fermentation broth.

In a thirteenth embodiment, the present disclosure provides the method of the eighth embodiment, wherein a portion of the first extractant becomes entrained in the second fermentation broth, the method further comprising:

at least partially extracting the entrained first extractant from the second fermentation broth with a second extractant by a second liquid-liquid extraction.

In a fourteenth embodiment, the present disclosure provides the method of any one of embodiments 3, 8, or 13, wherein the second liquid-liquid extraction is carried out through a second porous membrane.

In a fifteenth embodiment, the present disclosure provides the method of any one of embodiments 3, 8, or 13, wherein the second extractant is an alkane or a combination of alkanes.

In a sixteenth embodiment, the present disclosure provides the method of any one of the twelfth to fifteenth embodiments, the method further comprising:

at least partially extracting the fermentation inhibitor from the first extract with an aqueous solution by a fourth liquid-liquid extraction.

In a seventeenth embodiment, the present disclosure provides the method of embodiment 16, wherein the fourth liquid-liquid extraction is carried out through a fourth porous membrane.

In an eighteenth embodiment, the present disclosure provides the method of any one of embodiments 2, and 4 to 17, further comprising recovering at least a portion of the alcohol.

In a nineteenth embodiment, the present disclosure provides the method of any one of embodiments 1 to 18, wherein the first extractant has a solubility parameter of at least 7.5 (calories per cubic centimeter)^{1/2}.

In a twentieth embodiment, the present disclosure provides the method of any one of embodiments 1 to 19, wherein the first extractant has a solubility parameter in a range from 7.5 to 11 (calories per cubic centimeter)^{1/2}.

In a twenty-first embodiment, the present disclosure provides the method of any one of embodiments 1 to 20, wherein the first extractant has a solubility parameter in a range from 8.6 to 11 (calories per cubic centimeter)^{1/2}.

In a twenty-second embodiment, the present disclosure provides the method of any one of embodiments 1 to 21, wherein the solubility of the at least one of pentose or hexose sugars in the first extractant is less than one percent by weight.

In a twenty-third embodiment, the present disclosure provides the method of any one of embodiments 1 to 22, wherein the hydrolysis products from cellulosic biomass comprise pentose sugars, and wherein the microorganism is a pentose-fermenting microorganism.

In a twenty-fourth embodiment, the present disclosure provides the method of any one of embodiments 1 to 23, wherein the hydrolysis products from cellulosic biomass are produced by acidic, high-temperature steam treatment of corn stover.

In a twenty-fifth embodiment, the present disclosure provides the method of any one of embodiments 1 to 24, wherein the hydrolysis products from cellulosic biomass comprise at least one of acetic acid, lactic acid, formic acid, furfural, 5-hydroxymethylfurfural, or vanillin as the fermentation inhibitor.

In a twenty-sixth embodiment, the present disclosure provides the method of embodiment 25, wherein the hydrolysis products from cellulosic biomass comprise at least one of acetic acid, furfural, 5-hydroxymethylfurfural, or vanillin as the fermentation inhibitor.

In a twenty-seventh embodiment, the present disclosure provides the method of any one of embodiments 1 to 26, wherein the first extractant comprises a straight-chain or branched alcohol having from 8 to 20 carbon atoms.

In a twenty-eighth embodiment, the present disclosure provides the method of embodiment 27, wherein the first extractant comprises at least one of 2-ethyl-1-hexanol, 1-octanol, 2-octanol, 1-decanol, or 1-dodecanol.

In a twenty-ninth embodiment, the present disclosure provides the method of any one of embodiments 1 to 28, wherein the first extractant comprises at least one of acetophenone, 3-nonanone, ethyl butyrate, dibutyl maleate, 2-methyl-2-pentenal, or benzaldehyde.

In a thirtieth embodiment, the present disclosure provides the method of any one of embodiments 1 to 29, wherein the first porous membrane is a microporous membrane with pore sizes in a range from 0.07 micrometers to 1.4 micrometers.

In a thirty-first embodiment, the present disclosure provides the method of any one of embodiments 1 to 30, wherein the first porous membrane comprises polypropylene and has a thickness in a range from 1 micrometer to 100 micrometers.

In a thirty-second embodiment, the present disclosure provides the method of any one of embodiments 1 to 31, wherein the first liquid-liquid extraction is carried out in a liquid-liquid extraction element comprising:

a plurality of first layer pairs, each first layer pair comprising:
a first polymeric microporous membrane; and
a first flow channel layer oriented in a first flow direction having a first fluid inlet and a first fluid outlet disposed on first opposing sides of the liquid-liquid extraction element; and

a plurality of second layer pairs, with at least one second layer pair being disposed between two first layer pairs and at least one first layer pair being disposed between two second layer pairs so as to form a stack of layers, each second layer pair comprising:
a second polymeric microporous membrane; and
a second flow channel layer oriented in a second flow direction different than the first flow direction and having a second fluid inlet and a second fluid outlet disposed on second opposing sides of the extraction element.

In a thirty-third embodiment, the present disclosure provides a membrane solvent extraction system comprising:

a first vessel containing a volume of an aqueous mixture, the aqueous mixture comprising at least one of pentose or hexose sugars and a dissolved fermentation inhibitor that inhibits a microorganism otherwise capable of fermenting pentose or hexose sugars;

a second vessel containing a volume of a first extractant having a water solubility of less than one percent by weight;

a liquid-liquid extraction element comprising:
a plurality of first layer pairs in fluid communication with the first solution vessel, each first layer pair comprising:
   a first polymeric microporous membrane; and
   a first flow channel layer oriented in a first flow direction having a first fluid inlet and a first fluid outlet disposed on first opposing sides of the extraction element; and
a plurality of second layer pairs in fluid communication with the second vessel, with at least one second layer pair being disposed between two first layer pairs and at least one first layer pair being disposed between two second layer pairs so as to form a stack of layers, each second layer pair comprising:
   a second polymeric microporous membrane; and
   a second flow channel layer oriented in a second flow direction different than the first flow direction and having a second fluid inlet and a second fluid outlet disposed on second opposing sides of the extraction element;

wherein at least one of the first or second microporous membranes is disposed between the first flow channel layer and the second flow channel layer of each adjacent first layer pair and second layer pair, and wherein the fermentation inhibitor in the aqueous mixture can transfer into the first extractant across at least the first and second microporous membranes.

In a thirty-fourth embodiment, the present disclosure provides the membrane solvent extraction system of embodiment 33, further comprising:

a first entrance manifold in fluid communication with the first fluid inlet of each first layer pair and through which the aqueous mixture enters all of the first layer pairs;

a first exit manifold in fluid communication with the first fluid outlet of each first layer pair and through which a raffinate exits from all of the first layer pairs;

a second entrance manifold in fluid communication with the second fluid inlet of each second layer pair and through which the first extractant enters all of the second layer pairs; and

a second exit manifold in fluid communication with the second fluid outlet of each second layer pair and through which an extract exits from all of the second layer pairs.

In a thirty-fifth embodiment, the present disclosure provides the membrane solvent extraction system of embodiment 33 or 34, wherein the first extractant has a solubility parameter of at least 7.5 (calories per cubic centimeter)^{1/2}.

In a thirty-sixth embodiment, the present disclosure provides the membrane solvent extraction system of any one of embodiments 33 to 35, wherein the first extractant has a solubility parameter in a range from 7.5 to 11 (calories per cubic centimeter)^{1/2}.

In a thirty-seventh embodiment, the present disclosure provides the membrane solvent extraction system of any one of embodiments 33 to 36, wherein the first extractant has a solubility parameter in a range from 8.6 to 11 (calories per cubic centimeter)^{1/2}.

In a thirty-eighth embodiment, the present disclosure provides the membrane solvent extraction system of any one of embodiments 33 to 37, wherein the solubility of the at least one of pentose or hexose sugars in the first extractant is up to one percent by weight.

In a thirty-ninth embodiment, the present disclosure provides the membrane solvent extraction system of any one of embodiments 33 to 38, wherein the aqueous mixture comprises at least one of acetic acid, lactic acid, formic acid, furfural, 5-hydroxymethylfurfural, or vanillin as the fermentation inhibitor.

In a fortieth embodiment, the present disclosure provides the method of any one of embodiments 33 to 39, wherein the hydrolysis products from cellulosic biomass comprise at least one of acetic acid, furfural, 5-hydroxymethylfurfural, or vanillin as the fermentation inhibitor.

In a forty-first embodiment, the present disclosure provides the membrane solvent extraction system of any one of embodiments 33 to 40, wherein the first extractant comprises a straight-chain or branched alcohol having from 8 to 20 carbon atoms.

In a forty-second embodiment, the present disclosure provides the membrane solvent extraction system of embodiment 41, wherein the first extractant comprises at least one of 2-ethyl-1-hexanol, 1-octanol, 2-octanol, 1-decanol, or 1-dodecanol.

In a forty-third embodiment, the present disclosure provides the membrane solvent extraction system of any one of embodiments 33 to 42, wherein the first extractant comprises at least one of acetophenone, 3-nonanone, ethyl butyrate, dibutyl maleate, 2-methyl-2-pentenal, or benzaldehyde.

Objects and advantages of this disclosure are further illustrated by the following non-limiting examples, but the particular materials and amounts thereof recited in these examples, as well as other conditions and details should not be construed to unduly limit this disclosure.

### EXAMPLES

Unless otherwise noted, all parts, percentages, ratios, etc. in the Examples and the rest of the specification are by weight. These abbreviations are used in the following examples: g=grams, min=minutes, hr=hour, mL=milliliter, L=liter. If not otherwise indicated in the table, below, chemicals were obtained from Sigma-Aldrich, St. Louis, Mo.

Materials Table

| | |
|---|---|
| Acetic acid | EMD Chemicals, Darmstadt, Germany |
| Lactic acid | J T Baker, Phillipsburg, NJ |
| Formic acid and Furfural | Mallinkrodt, Paris, NY |
| 5-hydroxymethylfurfural (HMF) | Merck, Rahway, NJ |
| Glucose | GIBCO, Grand island, NY |
| 2-ethyl-1-hexanol and 3- | Alfa Aesar, Ward Hill, MA |
| nonanone | |
| Benzaldehyde | Eastman, Rochester, NY |
| Yeast extract | Bio-Springer, Maisons-Alfort Cedex, |
| | France |
| Bacto tryptone | Marcor, Carlstadt, NJ |
| Xylose | Fischer |
| Glucose and Fructose | ADM, Decatur, IL |
| KH₂PO₄ and K₂HPO₄ | Mallinckrodt, Phillipsburg, NJ |
| Carbenicillin | Carbenicillin Direct, UK |

### Example 1

Computer simulations of solvent properties and extraction efficiencies followed by confirmatory experimental extraction of synthetic inhibitor/sugar mixtures were performed. Computer simulations included computational chemistry molecular dynamics simulations (CERIUS 2/OFF, Accelrys, San Diego, Calif.) to calculate solvent solubility parameters and “ASPEN PLUS” process modeling simulations (Aspen Technology Inc, Burlington, Mass.) to simulate the extraction performance of various solvents for removing cellulosic fermentation inhibitors.

The computational chemistry molecular dynamics simulations employed the Dreiding Force Field. Solubility parameters were computed from the cohesive energy density after molecular dynamics simulations on periodic bulk samples of each solvent. The values of computed solubility parameters of various solvents are listed in Table 1.

The “ASPEN PLUS” modeling software modeled a standard liquid-liquid extraction column with streams of equal flow rate. The UNIQUAC properties package was selected for the simulation. The acid-pretreated cellulosic material (feed stream) was simulated to have the following inhibitor and sugar concentrations in mass percent: acetic acid 0.154%, lactic acid 0.369%, furfural 0.180%, formic acid 0.013%, hydroxymethylfurfural (HMF) 0.009%, levulinic acid 0.014%, dextrose 3.12%, and xylose 7.85%. The remainder of the feed stream consisted of water. The solvents were treated as pure for the purposes of modeling. Ten stages of extraction were used to allow for a good representation of each solvent for extraction performance.

In order to define a standardized method of comparing solvents, a new parameter called extraction yield was created. Extraction yield is a ratio of the concentration of a species in the extract to the concentration of that species in the feed. The definition of the extraction yield allows for values >1 when the concentration in the extract is greater than the concentration in the feed stream.

Another parameter, the extraction score, was defined in order to rank overall inhibitor removal performance. Each extraction yield of inhibitors and sugars was multiplied by a weighting factor determined by the importance of removing or retaining the particular compound in the feed. The resulting numbers were then summed to calculate the extraction score.

Water solubility values were obtained from the literature including IUPAC-NIST Solubility Data Series, Version 1, NIST Standard Reference Database 106, updated March 2006 available online at http://srdata.nist.gov/solubility/index.aspx; Lange's Handbook of Chemistry (16th edition), ed. Speight, James G, McGraw-Hill, NY, 2005; and Yaws'Handbook of Properties for Environmental and Green Engineering, ed. Yaws, Carl L, McGraw-Hill, NY, 2008. The calculated and simulated data is tabulated in Table 1.

The confirmatory extraction of a synthetic inhibitor/sugar mixture was next carried out using inhibitors and sugars to mimic the conditions in acid-pretreated cellulosic fermentation feed. The synthetic solutions contained 1 g/L each of formic acid, lactic acid, acetic acid, furfural, HMF and vanillin as well as 20 g/L of glucose and 60 g/L of xylose. Five mL of this synthetic solution and 5 mL of solvent were added to a test tube and the test tubes were shaken in a Lab Line Instruments (Mumbai, India) “MULTI-WRIST SHAKER” at a setting of 8 for 10 min at 20° C. The mixture was allowed to settle overnight to phase separate completely. Inhibitor concentrations in each phase were analyzed by an Agilent Technologies (Santa Clara, Calif.) 1100 HPLC system consisting of a quaternary gradient pump, autosampler, heated column compartment, and diode array absorbance detector. Gradient elution was done on a YMC Company Ltd. (Kyoto, Japan) “YMC Carotenoid S-5” (C30) column with 0.05% phosphoric acid and a solvent ramp of 0% acetonitrile/100% water to 90% acetonitrile/10% water and back over 32.5 minutes. Based on the measured concentration of each inhibitor and sugar, extraction yields and scores were calculated and shown in Table 1 and reported as “laboratory extraction yield” and “laboratory extraction score”; (see footnotes 2 and 3).

TABLE 1

| | | | | Extraction Yields | | | | | | | |
|---|---|---|---|---|---|---|---|---|---|---|---|
| | Solubility | Water | Simulated | Acetic | Lactic | Formic | | | | Xylose | Glucose |
| | parameter. | Solubility | Extraction | acid | acid | acid | Furfural | HMF | Vanillin | (C5) | (C6) |
| Extractant | (cal/cm³)^{1/2} | g/100 mL | Score | 5¹ | 2¹ | 2¹ | 5¹ | 3¹ | 5¹ | −100¹ | −50¹ |
| 3-methyl-3- | 9.1 | 0.50 | 6.00 | 0.255 | 0.191 | 0.080 | 0.937 | 0.421 | 0.936 | 0.0374 | 0.0550 |
| pentanol | | | | | | | | | | | |
| 2-hexanol | 9.4 | 0.35 | 10.9 | 0.159 | 0.082 | 0.043 | 0.956 | 0.494 | 0.955 | 0.0067 | 0.0096 |
| 2-ethyl-1- | 8.8 | 0.10 | 12.6 | 0.445 | 0.043 | 0.028 | 0.978 | 0.234 | 0.977 | 0.0015 | 0.0020 |
| hexanol | | | (11.1)² | 0.352³ | 0.112³ | 0.213³ | 0.669³ | 0.337³ | 0.918³ | | |
| 1-octanol | 9.0 | 0.11 | 11.1 | 0.125 | 0.214 | 0.033 | 0.961 | 0.288 | 0.961 | 0.0029 | 0.0038 |
| | | | (11.5)² | 0.361³ | 0.180³ | 0.239³ | 0.728³ | 0.360³ | 0.931³ | | |
| 2-octanol | 8.9 | 0.11 | 11.3 | 0.155 | 0.077 | 0.039 | 0.962 | 0.454 | 0.961 | 0.0043 | 0.0056 |
| | | | (11.4)² | 0.397³ | 0.155³ | 0.280³ | 0.687³ | 0.394³ | 0.937³ | | |
| 2,6-dimethyl- | 8.5 | 0.045 | 10.9 | 0.117 | 0.043 | 0.029 | 0.969 | 0.242 | 0.968 | .00016 | 0.0020 |
| 4-heptanol | | | (9.6)² | 0.301³ | 0.070³ | 0.180³ | 0.593³ | 0.220³ | 0.851³ | | |
| 1-decanol | 8.8 | 0.10 | 10.4 | 0.103 | 0.031 | 0.028 | 0.957 | 0.142 | 0.956 | 0.0017 | 0.0019 |
| | | | (10.5)² | 0.281³ | 0.120³ | 0.180³ | 0.666³ | 0.280³ | 0.911³ | | |
| 1-dodecanol | 8.5 | 0.10 | 10.4 | 0.077 | 0.018 | 0.018 | 0.983 | 0.058 | 0.972 | 0.0003 | 0.0002 |
| | | | (8.2)² | 0.000³ | 0.050³ | 0.020³ | 0.593³ | 0.200³ | 0.903³ | | |
| Acetophenone | 10.6 | 0.10 | 11.3 | 0.083 | 0.032 | 0.019 | 1.078 | 0.030 | 1.077 | 0.0004 | 0.0003 |
| | | | (12.5)² | 0.351³ | 0.137³ | 0.234³ | 0.917³ | 0.647³ | 0.700³ | | |
| 3-nonanone | 8.61 | 0.010 | 6.20 | 0.056 | 0.006 | 0.993 | 0.011 | 0.002 | 0.764 | 0.0000 | 0.0000 |
| | | | (12.0)² | 0.321³ | 0.030³ | 0.130³ | 0.895³ | 0.310³ | 0.931³ | | |
| 2-nonanone | 8.7 | 0.050 | 10.3 | 0.072 | 0.011 | 0.015 | 0.991 | 0.006 | 0.988 | 0.0000 | 0.0000 |
| isopropyl | 7.8 | 0.20 | 7.10 | 0.280 | 0.002 | 0.005 | 0.998 | 0.000 | 0.136 | 0.0000 | 0.0000 |
| ether | | | | | | | | | | | |
| ethyl butyrate | 9.5 | 0.50 | 12.3 | 0.374 | 0.007 | 0.243 | 0.997 | 0.009 | 0.996 | 0.0001 | 0.0000 |
| | | | (12.3)² | 0.221³ | 0.050³ | 0.160³ | 0.937³ | 0.360³ | 1.001³ | | |
| benzaldehyde | 11.0 | 0.01 | 11.8 | 0.357 | 0.015 | 0.022 | 1.006 | 0.008 | 1.006 | 0.0011 | 0.0009 |
| | | | (16.3)² | 0.301³ | 0.070³ | 0.200³ | 1.114³ | 0.841³ | 1.392³ | | |
| cyclohexane | 8.5 | 0.0017 | 4.2 | 0.018 | 0.000 | 0.001 | 0.813 | 0.000 | 0.002 | 0.0000 | 0.0000 |
| | | | (2.7)² | | | | | | | | |
| n-hexane | 7.7 | 0.10 | 3.30 | 0.022 | 0.000 | 0.001 | 0.635 | 0.000 | 0.002 | 0.0000 | 0.0000 |
| decahydro- | 8.5 | 0.002 | 2.80 | 0.006 | 0.000 | 0.001 | 0.561 | 0.000 | 0.001 | 0.0000 | 0.0000 |
| napthalene | | | | | | | | | | | |
| Oleyl alcohol | 8.4 | 0.018 | 8.7 | 0.059 | 0.009 | 0.013 | 0.974 | 0.014 | 0.700 | 0.0001 | 0.0000 |
| | | | (8.9)² | | | | | | | | |
| n-decane | 7.7 | 0.10 | 3.30 | 0.017 | 0.000 | 0.001 | 0.649 | 0.000 | 0.002 | 0.0000 | 0.0000 |
| | | | (2.1)² | | | | | | | | |
| n-dodecane | 7.6 | 0.10 | 2.70 | 0.009 | 0.000 | 0.001 | 0.538 | 0.000 | 0.001 | 0.0000 | 0.0000 |
| n-tetradecane | 7.5 | 0.10 | 2.60 | 0.007 | 0.000 | 0.001 | 0.511 | 0.000 | 0.001 | 0.0000 | 0.0000 |
| Dibutylamine | 8.0 | 0.35 | 5.10 | 0.082 | 0.024 | 0.028 | 0.910 | 0.000 | 0.239 | 0.0072 | 0.0086 |
| tripropylamine | 7.4 | 0.10 | 5.20 | 0.048 | 0.006 | 0.016 | 0.926 | 0.000 | 0.083 | 0.0013 | 0.0010 |
| Tributylamine | 7.5 | 0.30 | 4.80 | 0.022 | 0.002 | 0.004 | 0.904 | 0.000 | 0.023 | 0.0000 | 0.0000 |
| Trioctylamine | 7.4 | 0.10 | 3.60 | 0.014 | 0.001 | 0.003 | 0.690 | 0.000 | 0.009 | 0.0000 | 0.0000 |

¹= weighting factor for specific inhibitor
²= laboratory extraction score
³= laboratory extraction yield

### Example 2

Fifteen inhibitor compounds (Table 2) commonly found in heat (e.g., steam) and acid pre-treated corn stover were weighed out and dissolved in 12 liters of water to make up a master solution with target concentrations of approximately 0.1 wt % of each compound.

A 13 inch (83.9 cm)×13 inch (83.9 cm)×8 inch (20.3 cm) multi-layer cross-flow membrane module, similar to that described in U.S. Pat. App. Publ. No. 2007/011977 (Schukar et al.) and illustrated in FIG. 3, was constructed by stacking and edge-bonding laminate sheets of flat hydrophobic polypropylene thermally induced phase separation (TIPS) membrane/flat polypropylene spunbond/corrugated polypropylene film (corrugations serve as flow channels). As the stack was built up, sheets were placed so that each sheet had its flow channels oriented 90 degrees to the sheet above and below it in the stack. Layers were bonded to one another by applying a ⅜ inch (0.95 cm) wide bead of polybutylene hot melt to the corrugate grooves of the laminate, along the outer edges and then placing the next laminate sheet down into that hot melt, membrane side down. This formed a rigid module brick, with uniform faces; this brick contained 117 layers of these 13 inch (83.9 cm)×13 inch (83.9 cm) laminate sheets to form a membrane extraction module with a membrane surface area of approximately 11.8 m².

A housing unit was constructed to fit this module. The top and bottom of the housing unit were 13.5 inch×13.5 inch aluminum plates (¼″). 8.5-inch long corner posts, cut from 1-inch aluminum angle bar stock, were bolted to the bottom plate. When assembled, the top and bottom plate were parallel to one another and 8.5 inches apart. 1-inch vertical flanges were fixed to the outside perimeter of each plate for the purpose of attaching the fluid manifolds described below. The same 1-inch aluminum angle stock was used for this. A ¼-inch sheet of neoprene was placed between these plates and the top and bottom of the module, to prevent fluid bypass. To load the module “brick”, the top plate was removed, silicone adhesive was applied to the insides of the corner posts, and then the module was slid down into place between the posts, and seated against the lower neoprene sheet. The ¼-inch neoprene sheet was placed over the top of the module, and then the top plate was bolted to the posts.

Four 13.5 inch×11 inch outer diameter fluid manifolds (inlet and outlet for the aqueous phase/inlet and outlet for the solvent phase) were constructed from ⅜-inch

polycarbonate plates glued with outer ¾ inch thick, polycarbonate perimeter flanges (flange: 1 inch wide top and bottom, 1.5 inch on sides). The flange provided a fluid chamber opposite each module face that is 10.5 inch×9 inch×⅞ inch (with a ⅛ inch neoprene gasket). Ports (⅜ inch taps) were cut into the lower edges of each fluid manifold (centered in the edge of the ¾ inch polycarbonate perimeter flange). Corresponding vent ports were tapped into the top of each manifold. The aluminum corner posts were tapped on 3⅛ inch centers, and corresponding bolt holes were drilled through the faces of the manifold plates (centered in the flange). Manifolds were then bolted to the aluminum flanges, with ⅛ inch neoprene gaskets.

Twelve liters of 1-decanol as the extractant was pumped from a 22 liter storage tank to one face of the cross-flow module, through the module and then through a phase separator that removed any entrained aqueous phase, and returned to the storage tank. Likewise, the aqueous phase containing the inhibitor compounds, was pumped through the module (90 degrees to the direction of the solvent), through a gravity separator to remove entrained solvent and returned to its storage tank.

The 12 liter mixture of inhibitors was membrane extracted over a period of 8 hours. Extractant flow rate was 1 L/min, aqueous phase inhibitor mix flow rate was 1 L/min. Pressure on the aqueous phase was maintained at more than 5 cm water higher than the extractant phase, at all times during the experiment (maintained by back pressure valves on the exit manifolds of the housing unit). Samples of the solvent phase and the aqueous phase were taken at intervals during the run and analyzed using an “HP 6890” gas chromatograph (Agilent Technologies, Santa Clara, Calif.) with a DB-Wax 30 M Carbowax column, ID 0.53 mm (Agilent Technologies). Control samples of the individual compounds in water were used to identify peak locations for acetic acid, formic acid, lactic acid, furfural and 5-hydroxymethylfurfural. During the extraction then, areas under these five GC peaks were tracked as a function of time (Table 3). Areas under these peaks are proportional to the concentrations of the components. As Table 3 illustrates, over the 8 hour period of membrane extraction, there was a significant reduction in all five inhibitor components in the aqueous phase and a roughly proportional increase in concentration of these same components in the extractant phase.

TABLE 2

| Solvent | Grams added to 12 liters |
|---|---|
| acetic acid | 11.997 |
| formic acid | 13.667 |
| lactic acid | 14.131 |
| furfural | 12.013 |
| levulinic acid | 12.482 |
| 5-hydroxymethylfurfural (HMF) | 11.929 |
| 4-hydroxybenzoic acid | 11.945 |
| 4-hydroxybenzaldehyde | 12.032 |
| vanillic acid | 12.467 |
| syringic acid | 12.026 |
| vanillin | 11.938 |
| syringealdehyde | 12.024 |
| ferulic acid | 12.016 |
| isoferulic acid | 0.817 |
| 3,4-dihydroxybenzaldehyde | 12.438 |

TABLE 3

| | Aqueous | Aqueous final | Extractant | Extractant final |
|---|---|---|---|---|
| | initial peak | peak area | initial peak | peak area |
| | area (unless | (after 8 hrs | area (unless | (after 8 hrs |
| Inhibitor | indicated) | unless indicated) | indicated) | unless indicated) |
| acetic acid | 4593 | 2807 | NA | NA |
| lactic acid | 672 | 459 (3 hrs) | 664 (2.5 hrs) | 2307 (7 hrs) |
| furfural | 13115 | 5850 | 358 (40 min) | 5984 |
| levulinic acid | 1397 (40 min) | 1039 (2 hrs) | 657 | 900 (7 hrs) |
| 5-hydroxy- | 947 | 695 | 618 (6 hrs) | 942 (7 hrs) |
| methylfurfural | | | | |

NA = not analyzed

### Comparative Example 1

Corn stover (source: BioMass AgriProducts, Harlan Iowa) was acid pretreated by the US National Renewable Energy Laboratory (NREL, which is operated by the Midwest Research Institute for the US Department Of Energy) according to their technical report NREL/TP-510-42630 entitled “SSF experimental protocols: lignocellulosic biomass hydrolysis and fermentation”, laboratory analytical procedure (LAP), Oct. 30, 2001, N. Dowe and J. McMillan. The corn stover was mechanically ground and then processed in a pretreatment reactor at 30% solids with 1% (w/w) sulfuric acid and pretreated under pressure and a temperature of 180° to 200° C. The acid-pretreated corn stover was centrifuged to separate the liquid fraction and solid fraction. The liquid fraction contained mainly pentose sugars (C5 sugars), especially xylose, but it also contained some hexose sugars (C6 sugars), especially glucose. Cellulosic inhibitors (acids, furfural, HMF, vanillin, etc) were also present in the liquid fraction (referred to as C5 fraction).

There was no liquid-liquid extraction of the pretreated corn stover C5 fraction before fermentation.

An engineered E. Coli, FBR-5 strain, was used as C5 sugar fermentable microorganism in the fermentation. The FBR-5 strain, developed by the US Department of Agriculture (USDA, Peoria, Ill.), carries plasmid pLOI297, which contains the genes from Zymomonas mobilis necessary for efficiently converting pyruvate into ethanol as described in “Development of New Ethanologenic Escherichia coli Strains for Fermentation of Lignocellulosic Biomass”, Bruce S. Dien, et. al., Humana Press (2000).

A culture was grown overnight in 800 mL of filtered media, containing yeast extract 5 g, bacto tryptone 2 g, xylose 20 g, arabinose 5 g, glucose 5 g, galactose 2 g, fructose 0.4 g, K₂HPO₄ 12 g, KH₂PO₄ 4.9 g, sodium acetate 9.7 g, tetracycline 0.025 g, carbenicillin 0.025 g. The culture was inoculated with 0.5 mL frozen E. Coli FBR5 stock and grown for 17 hrs with a resulting optical density of 2.7. The complete volume was centrifuged in 2 sterile 500 mL centrifuge tubes for 6 min at 6000 rpm. The cell pellets were resuspended sequentially in 10 mL fresh media. 140 mL of the aqueous C5 fraction was inoculated with 3 mL of cell resuspension giving a starting optical density of 2.6. The volume of 140 mL was equally divided and transferred into two 100 mL media bottles with sample port, and CO₂ release for pH control with KOH or into 2 shake flasks with needle pierced rubber stoppers for pH control with solid calcium carbonate. The pH was maintained at 6.5. Fermentation was carried out at 35° C. in a shaker.

The amount of ethanol and sugar present in solution was estimated using High-Performance Liquid Chromatography (HPLC, Agilent 1200 obtained from Agilent Technologies) with an Aminex HPX-87H Column (obtained from Bio-Rad Laboratories Inc, Hercules, Calif.), a refractive index detector and 5 micromolar sulfuric acid in water as the mobile phase. Little fermentation was observed. The results are shown in Table 6, below.

### Comparative Example 2

As another comparative example, the C5 fraction after liquid/liquid solvent extraction (not membrane extraction) was tested for fermentation as in Comparative example 1. 250 mL of C5 fraction was combined with 250 mL extractant (1-decanol or dibutyl maleate) in a 600 mL separation funnel. After being shaken vigorously for 10 minutes it was left for 4 hrs for separation. The lower phase (C5 fraction) was decanted into a clean 500 mL media bottle. The funnel was emptied and refilled with 250 mL of fresh solvent, combined with the C5 liquid fraction and shaken for 10 minutes. Separation took place over about 12 hrs. The lower phase was decanted into a clean 500 mL media bottle and combined with 250 mL n-dodecane in a clean separator funnel. After shaking for 10 minutes it was left for separation for 1 hr. The lower phase (C5 fraction) was decanted into a clean 250 mL media bottle, which was then fermented as in Comparative Example 1. The amounts of sugars and ethanol were estimated by HPLC as described in Comparative Example 1. The results are shown in Table 6, below. Although the fermentation proceeded well the liquid/liquid solvent extraction resulted in significant emulsion formation and 10-20% or more yield loss.

### Example 3

In this example cellulosic inhibitors in the liquid fraction of the acid-pretreated corn stover of Comparative Example 1 were extracted with various extractants using a single layer flow-through membrane solvent extraction (MSE) module as shown in FIG. 3 of U.S. Pat. No. 7,122,709 (Fanselow et al.). Both sides of the 187 cm² membrane face metal channels of 1 mm height. Once the membrane was sandwiched, the MSE unit was vertically mounted in the system. Membrane has a thickness of 73 micrometers and average pore size of 0.32 micrometers.

The C5 fraction was pumped into the flow-through MSE unit in co-current mode with an extractant at 300 mL/min. Two pressure gauges were installed at both outlets. The intramembrane pressure was controlled during the operation such that aqueous phase pressure was 6.9 kPa (1 psi) higher than the extractant phase. Sampling from both aqueous and extractant phases was conducted throughout the 24 hour extractions and the samples were analyzed by HPLC as described in Example 1. Table 4 shows the initial and final concentration of inhibitors in the aqueous and extractant phases for the exemplary extractants, 1-decanol and dibutyl maleate.

TABLE 4

| | | |
|---|---|---|
| Solvent | 1-decanol | dibutyl maleate |

| | |
|---|---|
| Inhibitor | g/L in aqueous initial/final |
| | (g/L in extractant initial/final) |

| | | |
|---|---|---|
| Lactic acid | 4.31/4.06 | 4.12/4.31 |
| | (0.00/0.00) | (0.00/0.00) |
| Acetic acid | 8.65/5.14 | 7.28/6.83 |
| | (0.00/1.01) | (0.00/0.72) |
| 5-hydroxy- | 1.68/1.07 | 1.68/0.980 |
| methylfurfural | (0.00/0.25) | (0.00/0.50) |
| (HMF) | | |
| Furfural | 1.50/0.300 | 1.47/0.160 |
| | (0.00/0.43) | (0.00/0.90) |
| Vanillin | 0.130/0.001 | 0.130/0.005 |
| | (0.00/0.06) | (0.00/0.09) |

The aqueous phase was transferred into a clean 500 mL media bottle and combined with 250 mL n-dodecane in a clean separator funnel. After shaking for 10 minutes it was left for separation for 1 hr. The lower phase (C5 fraction) was decanted into a clean 250 mL media bottle, which was then fermented as described in Comparative Example 1. The amounts of sugars and ethanol were estimated by HPLC as described in Comparative Example 1. The results are shown in Table 6, below.

### Example 4

Example 3 was repeated but with a multilayer cross-flow MSE module similarly to that illustrated in FIG. 3 with module dimensions of 8 inch (20.3 cm)×8 inch (20.3 cm)×2 inch (5.1 cm) with 45 membrane layers and total membrane surface area of 14,200 cm². The porous membrane had a thickness of 75 micrometers and an average pore size of 0.39 micrometers. Pressure gauges were installed at both outlets. The intramembrane pressure and temperature were monitored and controlled during the operation. Sampling from both aqueous and solvent phases was conducted throughout the 8 hour extractions and samples were analyzed by HPLC as described in Example 1. Table 5 shows the initial and final concentrations of inhibitors in the aqueous and solvent phases for the various solvents tested.

TABLE 5

| | | |
|---|---|---|
| Solvent | 1-decanol | 2-octanol |

| | |
|---|---|
| Inhibitor | g/L in aqueous initial/final |
| | (g/L in solvent initial/final) |

| | | |
|---|---|---|
| lactic acid | 1.94/1.35 | 1.27/1.44 |
| | (0.02/0.04) | (0.00/0.02) |
| Acetic acid | 3.59/2.86 | 3.17/2.13 |
| | (0.12/0.73) | (0.04/0.52) |
| 5-hydroxy- | 0.74/0.55 | 0.71/0.44 |
| methylfurfural | (0.02/0.21) | (0.01/0.13) |
| (HMF) | | |
| furfural | 0.76/0.30 | 0.75/0.19 |
| | (0.09/0.52) | (0.05/0.27) |
| vanillin | 0.20/0.12 | NA |
| | (0.10/0.42) | |

The aqueous phase was transferred into a clean 500 mL media bottle and combined with 250 mL n-dodecane in a clean separator funnel. After shaking for 10 minutes it was left for separation for 1 hr. The lower phase (C5 fraction) was decanted into a clean 250 mL media bottle, which was then fermented as described in Comparative Example 1. The amounts of sugars and ethanol were estimated by HPLC as described in Comparative Example 1. The results are shown in Table 6, below.

TABLE 6

| | |
|---|---|
| Sugar (g/L) | Ethanol (g/L) |

| Solvent | 0 hrs | 24 hrs | 48 hrs | 0 hrs | 24 hrs | 48 hrs |
|---|---|---|---|---|---|---|
| CE1: | 43 | 42 | NA | 0 | 1 | NA |
| None | | | | | | |
| CE2: | 48 | 1 | NA | 0 | 23 | NA |
| 1-decanol | | | | | | |
| CE2: | 45 | 1 | NA | 0 | 20 | NA |
| Dibutyl | | | | | | |
| maleate | | | | | | |
| EX3: | 48 | 3 | NA | 0 | 20 | NA |
| 1-decanol | | | | | | |
| EX3: | 51 | 2 | NA | 0 | 19 | NA |
| dibutyl | | | | | | |
| maleate | | | | | | |
| EX4: | 50 | 30 | 12 | 0 | 12 | 18 |
| 1-decanol | | | | | | |

NA = not analyzed

All patents and publications referred to herein are hereby incorporated by reference in their entirety. Various modifications and alterations of this invention may be made by those skilled in the art without departing from the scope and spirit of this invention, and it should be understood that this invention is not to be unduly limited to the illustrative embodiments set forth herein.

1. A method of removing a fermentation inhibitor from an aqueous mixture of hydrolysis products from cellulosic biomass, the method comprising:
providing the aqueous mixture of hydrolysis products from cellulosic biomass, the hydrolysis products comprising at least one of pentose or hexose sugars and a fermentation inhibitor that inhibits a microorganism otherwise capable of fermenting at least one of pentose or hexose sugars; and
at least partially extracting the fermentation inhibitor from the aqueous mixture with a first extractant by a first liquid-liquid extraction through a first porous membrane to provide a first extract and a raffinate, the first extractant having a water solubility of less than one percent by weight,
wherein the raffinate has a lower concentration of the fermentation inhibitor than the aqueous mixture.

2. The method of claim 1, wherein a portion of the first extractant becomes entrained in the raffinate, the method further comprising:
at least partially extracting the entrained first extractant from the raffinate with a second extractant by a second liquid-liquid extraction to provide an second extract and a fermentable feed solution.

3. The method of claim 1, wherein the method is a method for producing an alcohol from an aqueous mixture of hydrolysis products from cellulosic biomass, the method further comprising:
combining the raffinate with the microorganism; and
fermenting the raffinate to produce the alcohol.

4. The method of claim 2, wherein the method is a method for producing an alcohol from an aqueous mixture of hydrolysis products from cellulosic biomass, the method further comprising:
combining the fermentable feed solution with the microorganism; and
fermenting the fermentable feed solution to produce the alcohol.

5. The method of claim 1, wherein the aqueous mixture comprises an insufficient amount of the microorganism to ferment the aqueous mixture.

6. A method for producing an alcohol from an aqueous mixture of hydrolysis products from cellulosic biomass, the method comprising:
fermenting the aqueous mixture to provide a first fermentation broth, the fermentation broth comprising:
a microorganism for fermenting at least one of pentose or hexose sugars;
hydrolysis products from cellulosic biomass, the hydrolysis products comprising at least one of pentose or hexose sugars and a fermentation inhibitor that inhibits the microorganism; and
the alcohol;
at least partially extracting the fermentation inhibitor from the first fermentation broth with a first extractant by a first liquid-liquid extraction through a first porous membrane to provide a first extract and a second fermentation broth, the first extractant having a water solubility of less than one percent by weight,
wherein the second fermentation broth has a lower concentration of the fermentation inhibitor than the first fermentation broth.

7. The method of claim 6, wherein a portion of the first extractant becomes entrained in the second fermentation broth, the method further comprising:
at least partially extracting the entrained first extractant from the second fermentation broth with a second extractant by a second liquid-liquid extraction.

8. The method of claim 7, wherein the second extractant is an alkane or a combination of alkanes.

9. The method of claim 1, wherein the first extractant has a solubility parameter of at least 7.5 (calories per cubic centimeter)^{1/2}.

10. The method of claim 1, wherein the first extractant has a solubility parameter in a range from 8.6 to 11 (calories per cubic centimeter)^{1/2}.

11. The method of claim 1, wherein the solubility of the at least one of pentose or hexose sugars in the first extractant is up to one percent by weight.

12. The method of claim 1, wherein the hydrolysis products comprise pentose sugars, and wherein the microorganism is a pentose-fermenting microorganism.

13. The method of claim 1, wherein the hydrolysis products comprise at least one of acetic acid, lactic acid, formic acid, furfural, 5-hydroxymethylfurfural, or vanillin as the fermentation inhibitor.

14. The method of claim 1, wherein the first extractant comprises a straight-chain or branched alcohol having from 8 to 20 carbon atoms.

15. The method of claim 1, wherein the first extractant comprises at least one of acetophenone, 3-nonanone, ethyl butyrate, dibutyl maleate, 2-methyl-2-pentenal, benzaldehyde, 2-ethyl-1-hexanol, 1-octanol, 2-octanol, 1-decanol, or 1-dodecanol.

16. The method of claim 1, wherein the first liquid-liquid extraction is carried out in a liquid-liquid extraction element comprising:
a plurality of first layer pairs, each first layer pair comprising:
a first polymeric microporous membrane; and
a first flow channel layer oriented in a first flow direction having a first fluid inlet and a first fluid outlet disposed on first opposing sides of the liquid-liquid extraction element; and
a plurality of second layer pairs, with at least one second layer pair being disposed between two first layer pairs and at least one first layer pair being disposed between two second layer pairs so as to form a stack of layers, each second layer pair comprising:
a second polymeric microporous membrane; and
a second flow channel layer oriented in a second flow direction different than the first flow direction and having a second fluid inlet and a second fluid outlet disposed on second opposing sides of the extraction element.

17. (canceled)

18. The method of claim 6, wherein fermenting is carried out in a fermenter, and wherein the second fermentation broth is returned to the fermenter.

19. The method of claim 6, wherein the alcohol is butanol, the method further comprising recovering butanol.

20. A method for producing butanol from an aqueous mixture of hydrolysis products from cellulosic biomass, the method comprising:
fermenting the aqueous mixture in a fermenter to provide a first fermentation broth, the fermentation broth comprising:
a microorganism for fermenting at least one of pentose or hexose sugars;
hydrolysis product from cellulosic biomass, the hydrolysis products comprising at least one of pentose or hexose sugars; and
butanol;
at least partially extracting the butanol from the first fermentation broth with an extractant by a liquid-liquid extraction through a porous membrane to provide an extract and a second fermentation broth, the extractant having a water solubility of less than one percent by weight, wherein the second fermentation broth has a lower concentration of the butanol than the first fermentation broth; and
returning the second fermentation broth to the fermenter.

21. The method of claim 20, wherein the extractant comprises a straight-chain or branched alcohol having from 8 to 12 carbon atoms.
